(19) United States
(12) Patent Application Publication
(10) Pub. No.: US 2006/0067913 A1
(43) Pub. Date: Mar. 30, 2006

(54) HCV therapy

(76) Inventors: Rupert Donald Holms, London (GB); Ravshan Inoyatovich Ataullakhanov, Moscow (RU)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/188,616

(22) Filed: Jul. 25, 2005

Related U.S. Application Data
(63) Continuation-in-part of application No. PCT/GB04/00330, filed on Jan. 27, 2004.

(30) Foreign Application Priority Data
Jan. 27, 2003 (GB) 0301879.3

(51) Int. Cl. A61K 38/21; A61K 38/10; A61K 38/08

(52) U.S. Cl. 424/85.4; 514/14; 514/17

(57) ABSTRACT

A peptide which has at least 80% identity to a fragment of Domain A or B of the Hepreceptor of ezrin, is used in the treatment of viral hepatitis.

### REFERENCE TO RELATED APPLICATION

This application is a continuation-in-part of International Patent Applicant No. PCT/GB2004/000330, filed Jan. 27, 2004.

### FIELD OF THE INVENTION

This invention relates to the therapy of Hepatitis C virus.

### BACKGROUND TO THE INVENTION

Hepatitis C Virus (HCV) infection is a major global healthcare problem. The World Health Organisation has estimated that 170 m people have been infected although few of them show symptoms yet because the disease has a long incubation period. Ten to twenty percent of these people are likely to suffer serious liver disease, such as cirrhosis or cancer. According to the US National Institutes of Health, HCV kills 10,000 Americans every year and is responsible for 17,000 patients waiting for liver transplants every year. There are still three to four million new HCV infections every year. Approximately 70% of those infected will develop liver disease and 1-5% cancer. There is no vaccine for HCV.

Existing therapy for HCV is based on alpha2 interferon or alpha2 interferon coupled to polyethylene glycol which is known as pegylated interferon or peginterferon. Serious side-effects and poor therapeutic benefit are associated with current methods of treatment. The usual course of treatment includes injections of 3 million units of recombinant alpha-interferon, 3 times per week over 12 months. In some studies, less than half of patients on interferon treatment gained any therapeutic benefit.

The antiviral drug Ribavirin is used in combination with interferon therapy to improve efficacy. Combination therapy improves the proportion of patients benefiting from treatment, if they are infected with HCV genotype-2 or genotype-3. However, only 40% of patients infected with HCV-1B, the most common genotype in the USA, respond to combination therapy.

It is well known that existing interferon treatment causes severe influenza-like symptoms, and side-effects with interferon plus Ribavirin are generally worse that the side-effects of interferon alone. Prolonged treatment with alpha-interferon is accompanied by the development of additional side-effects which get worse over the course of treatment. The majority of patients present complaints of weakness, irritability, insomnia, head and muscular pains, and arthralgia (joint pain) in the course of treatment. Frequent complications of interferon therapy include anemia, neutropenia, thrombocytopenia and alopecia. More rarely, patients develop one or more of hypo-thyroidism, pathologic damage of the capillaries, lupus, sarcoidosis and bullous injuries of the skin, depression and psychoses and also the serious polyorgan toxic effects.

U.S. Pat. No. 5,773,573A describes Human Ezrin Peptide (HEP1), a 14 amino acid synthetic peptide identical with the amino acid sequence from position 324 to 337 in the central alpha helical region of human ezrin (a cell membrane signal transduction protein of the ERM protein family). GB2354241A describes regulatory/unfolding peptides of ezrin. These peptides are immune amplifiers and can be used to treat viral disease.

### SUMMARY OF THE INVENTION

The present invention is based on the finding that either HEP1 (Gepon) or regulatory/unfolding peptides of Ezrin, e.g. when used in combination with interferons, for example alpha-interferon or peginterferon, may reduce the side-effects of interferon therapy and enhance interferon efficacy. Examples are provided, of successful therapy using HEP1, and also using combination therapy of recombinant alpha-interferon with HEP1 that reduced the side-effects of interferon, and increased the effectiveness of the antiviral treatment.

According to one aspect of the invention, HCV is treated by the administration of a peptide that comprises a sequence having at least 80% identity to a fragment of Domain A or B of the Hepreceptor of ezrin, and wherein the peptide binds to the Hepreceptor with at least as great affinity as HEP1.

According to another aspect of the invention, the peptide is used in the treatment of a patient who has viral hepatitis and is undergoing treatment with an interferon. The two agents may be given simultaneously, separately or sequentially.

### DESCRIPTION OF THE INVENTION

The peptide that is used in the invention preferably has 5 to 50 amino acids. It is or comprises a fragment of ezrin or closely related thereto. Thus, it may be, for example, TEKKRRETVEREKE (SEQ ID NO:1); see, for example, U.S. Pat. No. 5,773,573A (the contents of which are incorporated herein by reference). SEQ ID NO:1 corresponds to amino acids 324-337 of ezrin. Any peptide having at least 80%, preferably at least 90% identity (or 100% identity) to this sequence, or to any fragment of ezrin, and which has at least substantially the same activity, may be used. Suitable fragments of ezrin, from Domain A and B of the Hepreceptor, including EREKE, are described in GB2354241A and in U.S. patent application Ser. No. 09/856,070, filed May 17, 2001, the content of which is incorporated herein by reference (see in particular claims 6, 9 and 19).

The active agents used in the invention are known, or can be prepared by known methods. In particular, methods of synthesis of alpha-interferons and peptides such as HEP1, i.e. a peptide with the amino acid sequence TEKKRRETVEREKE, or regulatory/unfolding peptides of ezrin, are known by those skilled in the art.

The respective active agents may be administered in either order or simultaneously, e.g. in the same composition. They may be formulated together or independently, preferably in a form suitable for oral or parenteral administration.

Formulations, routes of combinations and dosages of the active components used in the invention are known, or can be determined, by those skilled in the art, based on the usual factors such as the age, height, sex and/or health of the patient. A skilled medical person can readily determine an effective dosage. By way of example, administration by mouth of 1 to 10 mg or by injection of 0.1 to 1 mg of HEP1 or a regulatory/unfolding peptide of ezrin, one to five times daily or up to 35 times weekly, is suitable, e.g. in combination with standard treatment protocols for interferon therapy.

The following Examples illustrate the invention. “HEP1” (also known as Human Ezrin Peptide 324-337 and Gepon) is also the peptide having SEQ ID NO:1.

### EXAMPLE 1

Eleven patients with chronic HCV infection (measured by HCV serum RNA PCR) and liver disease (measure by elevated levels of Asp and Ala transaminases), were treated with injections of recombinant alpha2 interferon plus 2 mg HEP1/5 ml water solution by mouth twice a day (Treatment Group). Ten patients with chronic HCV infection were treated with recombinant alpha2 interferon alone (Control Group).

The result was that in the Treatment Group there was at least a 20-fold enhancement in the suppression of HCV viral load compared to the Control Group and a reduction of transaminase levels to healthy levels (the Control Group still had significantly elevated level of transaminases in the blood). Further there was an unexpected and remarkable reduction in interferon side-effects in the Treatment Group, particularly in the areas of general pain, aching joints, bad taste in the mouth and weakness.

The data are shown in the following Table (Group Mean for each data point).

| Treatment group: | | | | Control Group: | | | |
|---|---|---|---|---|---|---|---|
| HEP1 + Interferon | | | | Interferon only | | | |
| 11 patients | | | | 10 patients | | | |
| | Month | Month | Month | | Month | Month | Month |
| Before | 1 | 2 | 3 | Before | 1 | 2 | 3 |

Symptoms
% of patient
group

| | | | | | | | | |
|---|---|---|---|---|---|---|---|---|
| Weakness | 55 | 27 | 18 | 27 | 50 | 50 | 70 | 80 |
| Pain | 82 | 36 | 18 | 18 | 70 | 80 | 70 | 70 |
| Bitter taste | 64 | 27 | 9 | 9 | 80 | 70 | 80 | 80 |
| aching joints | 27 | 36 | 18 | 18 | 0 | 0 | 20 | 90 |
| Virology + biochemistry | | | | | | | | |
| HCV_RNA Log | 3.55 | | | 1.36 | 3.5 | | | 2.2 |
| load | | | | | | | | |
| Ala Trans | 133.36 | 84.55 | 68.55 | 53.09 | 194.7 | 90.7 | 62.4 | 71.5 |
| Asp Trans | 108.82 | 62.09 | 50.27 | 43.36 | 97.1 | 72.2 | 53.7 | 63 |
| Immunology | | | | | | | | |
| Leucocytes | 7.13 | 6.15 | 5.55 | 4.73 | 6.09 | 4.7 | 4.11 | 4.51 |
| Lymphocytes | 27.73 | 33.36 | 33 | 32.82 | 26.3 | 29.1 | 31 | 37.5 |
| Monocytes | 8.27 | 6.82 | 7.09 | 6.45 | 5.4 | 5.1 | 6.4 | 4.7 |

Study Details

21 patients (12 women, 9 men) aged from 18 to 55 years, sick with chronic hepatitis C participated in the study at The Gastroenterological Center in the Clinical Infectious Hospital No 1, Moscow. The diagnosis of chronic hepatitis C was based on the presence of specific antibodies to HCV in the blood of the patients during at least 6 months. The presence of HCV RNA in the peripheral blood was determined by PCR (polymerase chain reaction), and in all patients in the trial were in the replication phase of HCV infection. According to clinical data, and to the results of a biochemical study of the blood, in the majority of patients a moderate activity of chronic hepatitis C disease was well established.

All patients who participated in this investigation were treated with a recombinant alpha interferon (reaferon) at a dose of 3 millions units, 3 times a week. In addition to the background interferon therapy, 11 patients were treated with HEP1, 2 mg orally (the preparation was dissolved in 5 ml of water, held 2-3 min in the mouth, then swallowed) once a day, daily during 3 months. Patients were divided into 2 groups, i.e. the Treatment Group (11 patients who obtained the combination of interferon antiviral therapy with HEP1) and the Control Group (10 people patients who obtained interferon antiviral therapy only).

Treatment within the framework of the investigation continued for 3 months. Subsequently, the treatment with interferon was continued up to 1 year, even in the cases where HEP1 treatment had resulted in the disappearance HCV RNA from the blood. For the duration of this study, all patients attended the designated doctor not less than 4 times, directly before beginning treatment, and 1, 2 and 3 months after the beginning of treatment. Within the same period, in addition to the clinical inspection, clinical and biochemical analyses of the blood were conducted, and viral load was assessed by HCV RNA in a semi-quantitative PCR assay. The final estimation of efficacy of the treatment was conducted 3 months after the beginning of treatment. The criteria for efficacy were prevalence of symptoms; frequency of an improvement (or normalization) in the activity of hepatic enzymes Al-AT, As-AT, gamma GT; frequency of improvement in the clinical indices of the blood; and results of PCR analysis of HCV RNA (viral replication). Statistical processing of the results of treatment was conducted with the use of a computer program called ‘Statgraphics’.

Initial Clinical Status of the Patients (Before Treatment)

Patients in both treatment and control groups prior to the beginning of treatment were similar in the presence and manifestation of the symptoms characteristic of the hepatitis disease of the hepato-biliary system. Complaints of pain in the right subcostal area were presented in 16 out of 21 (76%) patients. Bitterness in the mouth was perceived by 15 patients (71%). Express weakness, a sign of general asthenovegetative syndrome, appeared distinctly in 11 (52%) patients.

On inspection, only one of the subjects had established jaundice of the skin and sclera. Hepatomegalia and splenomegalia occurred in 20 (95%) and 17 (81%) respectively of the patients. Diffuse changes in parenchyma of the liver were registered with UZI in 20 (95%) of 21 patients. One patient had reactive pancreatitis. In certain cases, pathology of the gall bladder was observed, including thickening of the walls of the gall bladder in 9 patients, distortion of the bile duct in 6 patients, and gall stone formation in 1 patient.

Dynamics of Clinical Manifestations in the Course of Treatment

During the course of treatment, the presence and the manifestation of dyspeptic phenomena were evaluated (pain in the right subcostal area, bitterness in the mouth), asthenovegetative symptoms (weakness, irritability) and arthralgia. In the Control Group, the frequency of complaints of expressed general weakness grew from 50% to 80%, irritability from 0 to 100%, and arthralgia from 0 to 90%. Related deterioration in the health of patients during the course of interferon therapy can be explained by the widely known side-effects caused by all preparations of interferon.

In the Treatment Group, the frequency of complaints of general weakness was reduced from 54% to 18% (p<0.05), and arthralgia from 27% to 18% (p<0.1). Irritability in the first month of treatment was noted in 54% of patients, and in the subsequent months of treatment this index was reduced to 18% (p<0.05). These results demonstrated the positive influence of HEP1 on the side-effects and adverse reactions to interferon therapy in HCV patients.

In the majority of patients of both groups prior to the beginning of treatment, symptoms associated with liver disease were present. 70-80% of the patients noted pains in the right subcostal area and perceived bitterness in the mouth. In the Control Group, during the course of 3 months of treatment with interferon only, the frequency of complaints of pain in the right subcostal area and the bitterness in the mouth did not change. In the Treatment Group, the frequency of complaints of pain in the right subcostal area was reduced from 80% to 18% (p<0.05). After 2-3 months of treatment, bitterness in the mouth was perceived in only 9% of the Treatment Group, as against 64% in the Control (p<0.05) in comparison to the number of patients before treatment.

There were characteristic features of chronic hepatitis: hepatomegalia was noted by the doctor in attendance in 90-100% of patients, and splenomegalia in 70-90% of patients before treatment. In both groups of patients, treatment was equally effective in reducing hepatomegalia and splenomegalia. After the 3-month course of treatment, the frequency of hepatomegaly and splenomegalia was reduced to 30-40%.

Dynamics of the Level of the Hepatitis C Virus in the Blood

In all 21 patients investigated, a semi-quantitative PCR estimation of the level of HCV RNA in the blood was performed and the titre of HCV RNA varied from 10³ to 10⁴ at the start of the study. In the Control Group, after 3 months of treatment, in 7 (70%) of 10 people the titres of HCV RNA were reliably lower than the pretreatment levels. HCV RNA ceased to be detected in 2 (20%) of 10 patients. On average, the titre of HCV RNA was reduced by 1.3 log in the Control Group. Three patients in the Control Group (30%) failed to respond to interferon treatment and in one of these patients the level of virus increased 10 times during the course of interferon treatment.

In the Treatment Group, suppression of virus replication was far more effective than in the Control Group. All patients responded positively to the combination treatment and 10 out of 11 (91%) patients had a reliable reduction in the concentration of HCV RNA in the blood. The average titre of HCV RNA in the Treatment Group was reduced by 2.2 log, or 158 times, that is a reduction of viral load almost twenty times greater than in the Control Group. In 4 (36%) out of 11 patients in the Treatment Group, HCV RNA ceased to be detected. Not one patient of the Treatment Group registered an increase in the viral load.

Biochemical Indices of the Blood

The patients who participated in the study were sick with chronic hepatitis C, and suffered from a moderate increase in the level of Al-AT and As-AT in the blood, an indication of the destructive inflammation of the hepatocytes. In both groups, after only 1 month of treatment, a significant decrease in the level of Al-AT and As-AT in the blood occurred, which continued during the next month. In the Control Group, the levels tended to increase again in the third month. In the Treatment Group, there was a clear tendency toward a progressive and deeper reduction in Al-AT and As-AT. The average values of general bilirubin in both groups of patients were above the upper boundary of normal levels. In the course of treatment, the level of general bilirubin of the blood decreased by 15-20% to normal values.

Hematological Indices

The side-effects of prolonged therapy with recombinant interferon appear in changes in a number of indices of the blood. In the Control Group, a significant reduction in the maintenance of hemoglobin levels, the number of thrombocytes and especially in the quantity of mature neutrophilic granulocytes was observed, and simultaneously there was an increase of the content of lymphocytes, eosinophiles and macrophages/monocytes. In the Treatment Group, the combination of HEP1 with interferon prevented the majority of the pathological changes occurring, and the levels of neutrophils, granulocytes, eosinophils, lymphocytes and macrophages/monocytes remained at normal values during the 3 months of treatment.

### EXAMPLE 2

In this Example, Human Ezrin Peptide therapy comprised oral administration of a 2 mg/2 ml aqueous solution of HEP1 to HCV patients who were also infected with HIV. The patients were treated twice a day for 10 days followed by once a day for 20 days in the absence of any other anti-HIV or anti-HCV therapy. Blood samples from patients were analysed before treatment and 30 days after the end of the 30 day treatment period. 18 of 19 patients had detectable HCV RNA by PCR and all 18 patients (100%) responded to treatment with a reduction of viral load. The average viral load in the group was reduced by 100× (−2 logs) and in 6 of 18 patients the HCV RNA was undetectable 30 days after the end of the 30 day treatment period. The 8 out of 18 patients who had failed to clear HCV with earlier interferon/ribavirin treatment, responded to HEP1 therapy with a drop in HCV viral load. There was a reduction of ALT and AST liver transaminase enzymes in the serum in 13 out of the 19 patients and normalisation of enzymes levels was achieved in 4 of the 6 patients in which HCV RNA became undetectable. All HCV genotypes characterised in the study responded to treatment including the difficult to treat genotypes HCV-1a and HCV-1b. No side effects or adverse events were observed during treatment with HEP1. Supporting the safety of the peptide treatment in this group of double HIV+HCV infected patients, the CD4 cells increased in 18 of 19 patients on average by 70 cells per microlitre, the CD4/CD8 index improved, and HIV viral load also dropped in 3 of 4 patients in which HIV RNA was measured.

### EXAMPLE 3

The results in Example 2 confirmed the beneficial effect observed when, in a separate study, HEP1 was used as an oral mono-therapy to treat chronic HCV hepatitis in children. Seven children were given an oral dose of 1 mg twice-a-day for 28 days, and compared to nine children who were untreated for HCV infection for the one month period of the study. HEP1 therapy led to a progressive reduction in the pathologically elevated levels of ALT and AST aminotransferases, whereas the young patients in the control group suffered increases in ALT and AST levels. In the same study, disbacteriosis of the bowel was analysed in the children suffering from acute HCV infection and HEP1 was shown to correct the microfloral homeostasis disrupted by HCV disease. The concentration of HCV virus in the treatment group dropped at least 10× using a local HCV RNA PCR titration assay. No side-effects or adverse reactions to HEP1 were reported.

Details of the study reported in Example 3 can be found in the following references:
Cherednichenko T et al, Application of the pharmaceutical Gepon to the treatment of acute and chronic Viral Hepatitis in children, Infections in Children 2003; 1: 44-47; and
Cherednichenko T et al, Positive influence of pharmaceutical Gepon on the clinical laboratory indices and disbacteriosis of the bowel in children suffering from Viral Hepatitis, Russian Medical Journal 2003; 11: 468-471.

We claim:

1. A method for the treatment of a patient who has viral hepatitis, which comprises administering to the patient a therapeutically effective amount of a peptide that comprises a sequence having at least 80% identity to a fragment of Domain A or B of the Hepreceptor of ezrin, and wherein the peptide binds to the Hepreceptor with at least as great affinity as TEKKRRETVEREKE HEP1.

2. The method of claim 1, wherein the peptide comprises TEKKRRETVEREKE HEP1 or an active fragment thereof.

3. The method of claim 1, wherein the peptide comprises EREKE.

4. The method of claim 1, wherein the viral hepatitis is hepatitis C.

5. The method of claim 1, wherein the patient is additionally undergoing treatment with interferon.
